(19) United States
(12) Patent Application Publication
(10) Pub. No.: US 2025/0152537 A1
(43) Pub. Date: May 15, 2025

(54) COMPOSITIONS AND METHODS FOR THE TREATMENT OF HUMAN IMMUNODEFICIENCY VIRUS

(71) Applicant: PROTHIONE LLC, Franklin, TN (US)

(72) Inventors: Laura LILE, Franklin, TN (US); Albert CRUM, Franklin, TN (US)

(21) Appl. No.: 18/839,225

(22) PCT Filed: Feb. 15, 2023
(86) PCT No.: PCT/US2023/062649

§ 371 Date: Aug. 16, 2024

Related U.S. Application Data
(60) Provisional application No. 63/311,039, filed on Feb. 16, 2022.

(51) Int. Cl. A61K 31/198; A61K 9/00; A61K 31/122; A61P 31/18

(52) U.S. Cl. CPC A61K 31/198; A61K 9/0053; A61K 31/122; A61P 31/18

(57) ABSTRACT

Compositions and methods for inhibiting human immunodeficiency viral infection and for treating subjects already infected with a human immunodeficiency virus such as HIV-1. The compositions and methods reduce HIV viral load in subjects infected with HIV.

### FIELD OF THE INVENTION

The present disclosure generally relates to compositions and methods useful for reducing viral load in individuals infected with a human immunodeficiency virus (HIV) or at risk of becoming infected with a human immunodeficiency virus, including, for example, HIV-1.

### BACKGROUND

HIV belongs to the family of viruses known as retroviruses, which are a group of highly diverse, enveloped, positive-sense, and single-stranded RNA viruses that include HIV-1 and HIV-2. The more common strain of HIV, HIV-1, accounts for more than 95% of all infections worldwide and is more transmissible than HIV-2.

Similar to other viruses, HIV causes infections by going through a viral replication cycle. After entering into the host cell, the virus releases nucleic acid and forces the cell to replicate the viral genome. Transcription and translation subsequently occurs for protein synthesis and assembly of viral components. The newly formed virus is released from the host cell to extracellular space. The viral load can cause pathogenesis after increasing to a certain point. The common symptoms of HIV can include fever, dry and itchy skin or skin infections, rashes, cough, sweats, weight loss, diarrhea, and if the infection goes untreated, can lead to acquired immunodeficiency syndrome (AIDS).

Although an HIV infection can be treated through antiretroviral therapy (ART) to the point of viral loads being undetectable, it is possible for HIV viral loads to increase and for CD4 cell count to decrease due to inconsistency in medicine usage or biological incompatibility with the medications prescribed. When HIV reproduces in an uncontrolled manner, viral levels can increase to the point of damaging the immune system, allowing for opportunistic infections. In the event of disease progression, an HIV patient receives an AIDS diagnosis when the patient's CD4 cell count falls below 200 cells/mm or the patient develops an opportunistic infection, during which the patient is highly infectious and can easily transmit the virus through certain bodily fluids, during pregnancy, birth, or breastfeeding, etc. The global fatality rate due to HIV/AIDS was 1.7% in 2017, although in some countries, the share of HIV/AIDS fatality rates was much higher. For example, 28% of deaths in South Africa and Botswana were caused by HIV/AIDS that year. HIV is one of the world's most fatal infectious diseases and has had severe impacts on health and on the global economy since the epidemic began in the 1980s.

There are 24 FDA-approved therapeutics, plus 23 combination therapeutics for the treatment of HIV. However, due to the nature of mutations in the human immunodeficiency virus during replication, these medicines are often combined to increase efficacy, with three HIV medications from at least two different HIV drug classes often prescribed. Given the possibility of ineffective therapeutics, which can be due to the virus' mode of replication, the patient's biological incompatibility with certain medications, or error in the patient's administration of the therapeutics, there is a need for new treatment options for HIV-infected individuals.

### SUMMARY OF THE INVENTION

Described herein are compositions and methods that are useful in treating subjects infected with a human immunodeficiency virus, such as HIV-1 or HIV-2. The compositions and methods are also useful as a prophylaxis, to reduce the risk of becoming infected and/or of developing severe illness if infected with a human immunodeficiency virus, such as HIV-1 or HIV-2. The methods and compositions described herein are effective in raising intracellular glutathione levels in virus-infected individuals. Administering a composition as described herein to HIV patients also reduces HIV viral load, and thereby can reduce patients' infectivity and spread of the virus.

The compositions described herein generally comprise glycine, cystine, and a glutamate source; such components provide the amino acids required for glutathione synthesis. In certain embodiments, the compositions also comprise a selenium source and coenzyme Q10. Such compositions, when administered to subjects infected with a human immunodeficiency virus or at risk of becoming infected with a human immunodeficiency virus, efficiently deliver to cells components for glutathione synthesis, thereby raising intracellular glutathione levels and/or inhibiting the depletion of intracellular glutathione that can result from a viral infection.

The present disclosure relates to these and other important aspects.

### BRIEF DESCRIPTION OF THE DRAWINGS

FIG. 1 is a bar graph showing the mean number of days for HIV-positive study subjects to reach clinical resolution of coronavirus disease 2019 (COVID-19), as described in the Examples. For HIV-positive study subjects in the Prothione™-treatment arm, the mean number of days to reach clinical resolution was 10 days, whereas in the placebo arm, the mean number of days to reach clinical resolution was 16.67 days.

FIG. 2 shows graphs for individual HIV-positive study subjects, with Clinical Symptom Score Assessment on the y-axis and days on the x-axis. The graph on the left is for study subject PID 257 in the Prothione™-treatment arm; the graph on the right is for study subject PID 356 in the placebo arm. The star on each graph indicates the day on which the study subject reached clinical resolution of COVID-19, as indicated by three negative RT-PCR tests (for SARS-COV-2), with each of the last two tests conducted within 24-36 hours of the previous test.

FIG. 3, similar to FIG. 2, shows graphs for individual HIV-positive study subjects, with Clinical Symptom Score Assessment on the y-axis and days on the x-axis. The graph on the left is for study subject PID 001 in the Prothione™-treatment arm; the graph on the right is for study subject PID 343 in the placebo arm. The star on each graph indicates the day on which the study subject reached clinical resolution of COVID-19, as indicated by three consecutive negative RT-PCR tests (for SARS-COV-2) as described herein.

FIG. 4 is a line graph showing HIV viral load, as measured in two HIV-positive study subjects (PID 234 and PID 368) in the Prothione™-treatment arm.

FIG. 5 is a line graph showing HIV viral load, as measured in an HIV-positive study subject (PID 046) in the placebo arm.

### DETAILED DESCRIPTION OF THE INVENTION

The features and advantages of the present invention may be more readily understood by persons of ordinary skill in the art upon reading the following detailed description. It is to be appreciated that certain features of the invention that are described above and below in the context of separate embodiments may also be combined to form a single embodiment. Conversely, various features of the invention that are described in the context of a single embodiment for reasons of brevity may also be combined so as to form sub-combinations thereof. In addition, the drawings and specific embodiments of the invention described herein are illustrated by way of example, it being expressly understood that the description and drawings are only for the purpose of illustration and that the specific embodiments are not intended to define the limits of the present invention.

The present disclosure relates to methods of treating, and to methods of preventing severe symptoms of viral infections such as HIV infections. In general, the methods include administering to a subject a composition that increases the levels of intracellular glutathione. Such subject may be a person infected by HIV-1 or at risk of becoming infected with HIV-1. For example, the compositions described herein may be administered to individuals who have tested positive for HIV-1, or who have been exposed to bodily fluid(s) from someone who has HIV-1.

Viral infections can deplete glutathione levels within cells. Lee C., Therapeutic modulation of virus-induced oxidative stress via the Nrf2-dependent antioxidative pathway, Oxid Med Cell Longev. 2018:6208067; Ivanov AV et al., Oxidative Stress during HIV Infection: Mechanisms and Consequences. Oxid Med Cell Longev. 2016:8910396; Zhang Z et al., Flaviviridae Viruses and Oxidative Stress: Implications for Viral Pathogenesis, Oxid Med Cell Longev. 2019:1409582; Polonikov, A., Endogenous deficiency of glutathione as the most likely cause of serious manifestations and death in COVID-19 patients, ACS Infect Dis 6(7): 1158-1562 (2020). Depleted levels of reduced glutathione (GSH) and oxidative stress may play a role in the pathogenesis of viral infections and the development of severe disease. Embodiments of the present invention relate to compositions and methods for raising intracellular GSH levels in virus-infected cells, by administering to HIV-infected individuals components for GSH synthesis. As demonstrated herein, administering components for GSH synthesis to HIV-positive individuals reduces patients' HIV viral load, including when compared to HIV-positive individuals administered a placebo. The present disclosure therefore relates to compositions and methods useful in the treatment of HIV infections, including HIV-1 infections, and to compositions and methods useful as a prophylaxis in individuals at risk of being infected and/or at risk of developing severe disease if infected.

Glutathione is a tripeptide of glycine, cysteine, and glutamic acid. Under normal physiological conditions, glutathione is present in cells in relatively high concentrations. See, e.g., van't Erve, Thomas J. et al., The concentration of glutathione in human erythrocytes is a heritable trait, Free Radic Biol Med., 65:742-749 (2013), which summarizes GSH levels reported in the literature and provides an estimated intracellular GSH concentration range of 0.4 to 3.0 mM (mean 1.4 mM). Glutathione maintains a reduced intracellular environment that protects the cell from oxidative stress. The thiol group in the cysteine of glutathione is a reducing agent and can be reversibly oxidized and reduced.

In addition to its protective role as an antioxidant, GSH also may block viral entry into cells and viral replication. Fraternale A. et al., GSH and analogs in antiviral therapy, Mol Aspects Med. 30:99-110 (2009). While not wishing to be bound by theory or mechanism: the cysteine residue of a glutathione molecule may be capable of inhibiting the replication of viruses that rely on Zn²⁺-binding proteins; the cysteine may serve as a binding site for the zinc in viral zinc finger proteins that are critical for the viral life cycle. By sequestering metals that the virus requires for replication and survival, the sulfhydryl of the cysteine in glutathione may protect a host cell from viral challenge.

Restoring virally-depleted GSH back to normal levels is challenging. For example, supplying GSH directly to cells is not a viable option, for several reasons. GSH synthesis is subject to negative feedback inhibition, such that supplying GSH to cells can halt native GSH synthesis and could lead to a dangerous rebound effect in patients. Ballatori N. et al., Glutathione dysregulation and the etiology and progression of human diseases, Biol Chem. 390(3):191-214 (2009). In addition, the half-life of GSH in the blood is on the order of only seconds to minutes. Lu S C, Regulation of glutathione synthesis, Curr Top Cell Regul. 36:95-116 (2000). Further, any GSH that remains in the blood must overcome thermodynamic and biochemical hurdles to enter the cell: for example, GSH is membrane-impermeable.

Instead of supplying GSH to cells, supplying cysteine, or a precursor of cysteine, N-acetyl cysteine (NAC), to cells has been explored. However, cysteine and NAC may compete with glutathione for resources in certain GSH recycling pathways, such that loading a cell with cysteine or with NAC may result in less efficient recycling of glutathione. In addition, NAC must be enzymatically de-acetylated before cysteine is made available for use in glutathione synthesis, and thus NAC is not able to increase intracellular GSH levels efficiently. Further, almost one-third of an NAC dose may be excreted by the kidneys. Sansone R A, Sansone L A, Getting a knack for NAC: N-Acetyl-Cysteine, Innov Clin Neurosci. 8(1):10-14 (2011).

In cases where increased GSH levels are desired, such as in patients infected with a human immunodeficiency virus, a cystine-based product that includes the amino acid precursors for glutathione synthesis can be efficient at raising GSH levels, at least because there is no need for enzymatic de-acetylation (unlike for NAC).

Cystine can be rapidly converted to cysteine, which is the rate-limiting component of glutathione synthesis. Yildiz D. et al., Comparison of N-acetyl-L-cysteine and L-cysteine in respect to their transmembrane fluxes, Biochem Suppl Ser Membr Cell Biol. 3:157-162 (2009). Cystine is the natural extracellular reservoir of cysteine. Compared to cysteine, cystine has a longer half-life in the oxidized environment of the blood, and for at least some cell types it can readily move across the cell membrane. In addition, unlike for NAC, a cell's conversion of cystine to cysteine does not require an enzymatic reaction. Upon cellular entry, one molecule of cystine is reduced to two cysteines that are immediately available for GSH synthesis. The addition of selenium also may be beneficial to patients with GSH depletion, as selenium serves as a cofactor in GSH biosynthesis. Selenium deficiency also has been tied to COVID-19 outcomes. Moghaddam A. et al., Selenium deficiency is associated with mortality risk from COVID-19, Nutrients 12(7):2098 (2020).

Embodiments of the present invention generally relate to compositions comprising glycine, L-cystine, and a glutamate source. Such components provide the amino acids required for GSH synthesis. In certain embodiments, the glutamate source is glutamine and/or glutamic acid. In additional embodiments, the compositions further comprise a selenium source such as, for example, selenomethionine, selenocysteine, selenite, methylselenocysteine, and/or selenium nanoparticles. In certain embodiments, the amount of selenium source present in the composition, or the amount of selenium source that is administered to a subject, is sufficient to provide a dose of about 0.01 micrograms to about 20 micrograms of selenium. In further embodiments, the compositions also comprise coenzyme Q10.

In some embodiments, the compositions comprise glycine, L-cystine, a glutamate source, a selenium source, and coenzyme Q10. In certain such embodiments, the glutamate source is L-glutamine, and in further such embodiments, the selenium source is selenomethionine.

In any of the embodiments described herein, the glycine, cystine, and glutamate source (e.g., L-glutamine or L-glutamate) may be present in the composition as free-form amino acids. In addition, in any of the embodiments of the compositions and methods described herein, the stoichiometric ratio of glycine:cystine:glutamate administered to a subject can vary, e.g., from about 4:1:4 to about 1:4:1. In certain embodiments, the stoichiometric ratio is about 1:0.5:1.

In some embodiments, the present disclosure relates to a composition comprising glycine, L-cystine, a glutamate source (such as, e.g., L-glutamine or L-glutamic acid), and a selenium source (such as, e.g., selenomethionine, selenocysteine, or selenium particles), and optionally coenzyme Q10, for use in treating HIV-1, or for reducing HIV-1 viral load in a subject.

In certain embodiments, the composition comprises glycine, an L-glutamate source, L-cystine, and L-selenomethionine. In certain embodiments, the composition further comprises coenzyme Q10. In various embodiments, the composition further comprises a metallothionein or a fragment thereof.

In any of the various embodiments described herein, the composition may further comprise an additional agent that functions as a metal chelator. Such additional agent may be an Fe³⁺ chelator, a Zn²⁺ chelator, an Ni²⁺ chelator, or a combination thereof. The additional agent should be bio-compatible, and in some embodiments it may be desirable that the additional agent have a dissociation constant that is lower than the dissociation constant of relevant proteins (e.g., viral zinc finger proteins) that bind to the metal ions. Such agents may include, for example, zinc chelators such as N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy) ethane-N,N,N′,N′-tetraacetic acid (BAPTA), and ethylenediamine-N,N′-diacetic-N,N′-di-β-propionic (EDPA), etc. and iron chelators such as diethylene triamine pentaacetic acid (DETAPAC), dipyridyl, pyridoxal isonicotinoyl hydrazone (PIH), desferrioxamine (DFO), deferiprone (DFP) and deferasirox (DFS).

In certain embodiments, the present disclosure relates to use of a composition comprising glycine, L-cystine, a glutamate source selected from glutamine and glutamic acid, and a selenium source, optionally together with coenzyme Q10, for the manufacture of a medicament for treating HIV-1, or for reducing HIV-1 viral load in a subject. In some embodiments, the composition comprises a glycine, L-cystine, an L-glutamate source, L-selenomethionine, and coenzyme Q10.

A selenium source may comprise an inorganic selenium compound, e.g., an aliphatic metal salt containing selenium in the form of selenite or selenate anions, or an organic selenium compound, e.g., selenium cystine, selenium methionine, mono-or di-seleno carboxylic acids comprising about seven to eleven carbon atoms in the chain, or a seleno amino acid chelate. For a composition comprising glycine, L-cystine, and a glutamate source (e.g., glutamic acid or glutamine), the composition makes available two L-cysteines from the disulfide bond of L-cystine. When the composition also comprises L-selenomethionine, the composition further makes available an additional L-cysteine via transsulfuration of the methionine moiety in the selenomethionine. As L-cysteine is rate limiting for biosynthesis of glutathione, such compositions provide three L-cysteines, as well as the other amino acids needed for the synthesis of glutathione.

Example compositions include Immune Formulation 200™, which is a formulation of free-form amino acids and which comprises cystine, glycine, a glutamate source, and selenium. Immune Formulation 200™ has a favorable safety profile and is designed to overcome the hurdles discussed above for raising intracellular GSH levels. See generally US 2012/0029082; U.S. RE39,734; U.S. RE42,645; WO 2021/263206. In embodiments of methods as described herein, Immune Formulation 200™ can be administered with coenzyme Q10.

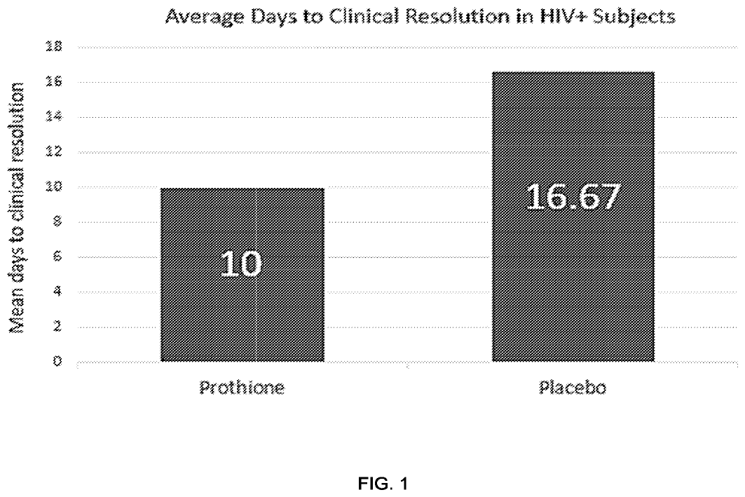

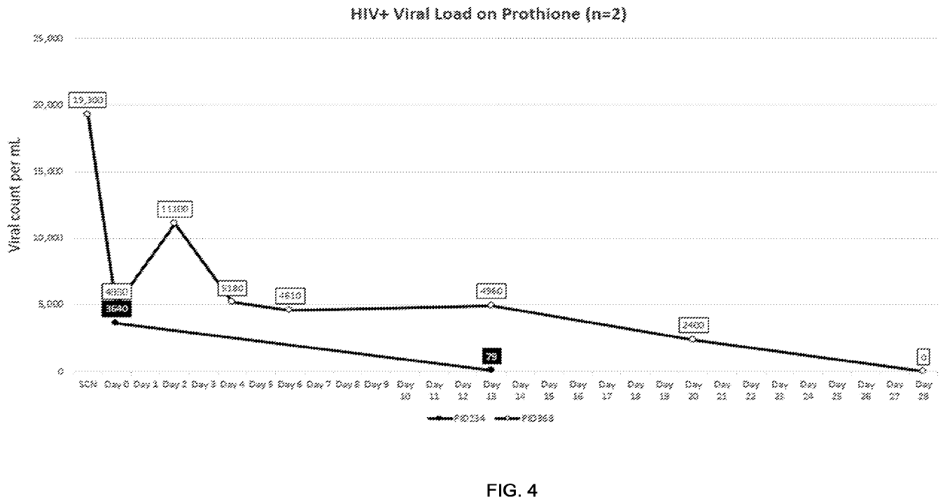

Another example composition is Prothione™. Prothione™ capsules comprise glycine, L-cystine, L-glutamine, selenomethionine, and coenzyme Q10. In the study described in the Examples, it was demonstrated that administering a Prothione™ composition to patients diagnosed with COVID-19 increases intracellular glutathione and reduces the duration and severity of the disease. When compared to COVID-19 patients taking placebo, COVID-19 patients taking Prothione™ showed a noticeable reduction in time to clinical resolution, which was defined as the time (in days) to attain three consecutive negative RT-PCR tests as described herein; this result was also observed in study subjects who were also HIV-positive (see FIG. 1). Patients treated with Prothione™ also showed a significant decrease in viral load of COVID-19, when compared to patients taking placebo. It was further demonstrated that, in study subjects who were also HIV-positive, administering Prothione™ reduced the HIV viral load (see FIG. 4).

Administering compositions comprising glycine, cystine, a glutamate source, and optionally further comprising a selenium source and coenzyme Q10 has several advantages over other anti-viral therapies. For example, the compositions described herein can be safely administered to subjects of all age groups (including, e.g., young children who may be at risk of exposure to HIV-1), as the dose of the composition can be selected such that the amount of selenium (if present in the composition) administered is below the selenium upper intake limit for infants and children (as set by the Food and Nutrition Board at the Institute of Medicine of the National Academies). There is also little concern for side-effects or drug interactions.

Embodiments of the invention relate to methods of administering, to a subject diagnosed with HIV-1 or to a subject exposed to HIV-1, for example, the following: 1177.5 mg glycine, 600.3 mg L-cystine, 1177.5 mg L-glutamine, 6 mg coenzyme Q10, and 0.017 mg selenomethionine. In certain such embodiments (such as for pediatric patients), the 1177.5 mg glycine, 600.3 mg L-cystine, 1177.5 mg L-glutamine, 6 mg coenzyme Q10, and 0.017 mg selenomethionine are administered once daily, and in other embodiments (such as for adult patients), the 1177.5 mg glycine, 600.3 mg L-cystine, 1177.5 mg L-glutamine, 6 mg coenzyme Q10, and 0.017 mg selenomethionine are administered twice daily (e.g., in the morning and in the evening). It should be understood that the amounts described herein are approximations and encompass a range within limits typically accepted in the pharmaceutical industry.

In further embodiments, a patient diagnosed with HIV-1, or an individual exposed to HIV-1 or suspected of having HIV-1, is administered Prothione™. In some embodiments, Prothione™ is administered as three 1-gram Prothione™ capsules, either once daily (for a total daily dose of 3 grams Prothione™) or twice daily (for a total daily dose of 6 grams Prothione™). For certain embodiments involving pediatric subjects under 3 years of age and/or under 40 kg in weight, three 1-gram Prothione™ capsules (or other dosage form such as a solution or suspension comprising the same amounts of glycine, L-glutamine, L-cystine, coenzyme Q10, and selenomethionine as are present in three 1-gram Prothione™ capsules) are administered once daily. In alternative embodiments involving pediatric subjects (e.g., patients at least 3 years of age and under 40 kg in weight), a pediatric subject is administered 588 mg glycine, 588 mg L-glutamine, 300 mg L-cystine, 3 mg coenzyme Q10, and 0.0085 mg selenomethionine twice daily (e.g., in the morning and in the evening).

In some embodiments, the methods described herein further comprise

administering to a subject a metal chelator, in addition to the glycine, cystine, glutamate source, and in some embodiments also the selenium source and coenzyme Q10. The metal chelator may be an Fe³⁺ chelator, a Zn²⁺ chelator, an Ni²⁺ chelator, or a combination thereof. The metal chelator may be included in any of the compositions described herein, or may be administered separately.

In some embodiments, the methods comprising administering to a subject glycine, cystine, a glutamate source, and optionally a selenium source and coenzyme Q10 as described herein, further comprise administering to the subject a metallothionein or fragment thereof.

In certain embodiments, the methods described herein (such as a method of administering a composition as described herein to a subject) increase the subject's intracellular levels of reduced glutathione (GSH). In some embodiments, the methods described herein elevate the subject's intracellular concentration of glutathione by at least about 25%, at least about 30%, at least about 40%, at least about 50%, at least about 60%, at least about 70%, at least about 80%, at least about 90%, at least about 100%, at least about 120%, at least about 150%, or at least about 200%, compared to the subject's pre-treatment level of intracellular glutathione. In various embodiments, administering a composition as described herein to a subject elevates the subject's intracellular concentration of glutathione by at least about 40%. In various embodiments, administering a composition as described herein to a subject allows the subject to reach an intracellular concentration of glutathione that is from 0.1 mM to 4.0 mM, by 24-48 hours following the first administration of the composition. For example, in certain embodiments an intracellular concentration of glutathione that is from 0.5 mM to 3.0 mM is reached by 24 hours after the first administration. In various embodiments, the administration of the composition to a subject is effective in allowing the subject to reach an intracellular concentration of glutathione that is from 2 mM to 4 mM, by 72 hours post-first administration. In some embodiments, administering a composition as described herein to a subject results in an intracellular concentration of glutathione that is at least 0.3 mM, at least 0.4 mM, at least 0.5 mM, at least 0.6 mM, at least 0.7 mM, at least 0.8 mM, at least 0.9 mM, at least 1.0 mM, at least 1.5 mM, at least 2.0 mM, at least 2.5 mM, at least 3.0 mM, at least 3.5 mM, or that is 4.0 mM, by 48 hours following the first administration of the composition.

In certain embodiments, a composition as described herein is administered at a dose and frequency that is effective to reduce or inhibit depletion of intracellular glutathione levels in coronavirus-infected cells at 24-72 hours following the first administration of the composition. In some embodiments, administering a composition as described herein is effective to restore intracellular glutathione levels in HIV-infected cells to the intracellular glutathione levels in non-infected cells at 24-48 hours following the first administration of the composition. A first administration of the composition may be before or after infection with a human immunodeficiency virus, or before or after exposure to a bodily fluid from someone infected with such a virus. In certain embodiments, the composition is first administered after infection with a human immunodeficiency virus (e.g., HIV-1), and in some embodiments is first administered after an exposure to a bodily fluid from someone infected with the virus. In specific embodiments, the composition is first administered from about 12 hours to about 96 hours post-infection with a human immunodeficiency virus (or similarly may be administered from about 12 hours to about 96 hours post-exposure). For example, the first administration of the composition to a subject may be from about 24 hours to about 72 hours post-infection with a human immunodeficiency virus (or similarly, from about 24 hours to about 72 hours post-exposure). In various embodiments, the composition is first administered about 48 hours post-infection with a human immunodeficiency virus (or similarly, about 48 hours post-exposure).

As described above, a “first” administration need not be the first time a subject has ever been administered the composition; rather, first administration refers to the first dose or first administration in a given series of administrations (e.g., for a treatment schedule involving twice-daily administration for ten days, the first administration would be the earlier (e.g., morning) administration occurring on day 1 of those ten days, even if the subject had been administered the composition a week prior to day 1).

In certain embodiments, administering a composition as described herein to a subject having HIV-1 reduces the subject's viral load by at least about 10%, at least about 15%, at least about 20%, at least about 25%, at least about 30%, at least about 40%, at least about 50%, at least about 60%, at least about 70%, at least about 80%, at least about 90%, or at least about 95%, compared to the subject's viral load at baseline. In some embodiments, administering a composition as described herein to a subject having HIV-1 reduces the subject's viral load by at least about 50% or more. In specific embodiments, the composition comprises glycine, L-cystine, L-glutamine, selenomethionine, and coenzyme Q10, each present in about the same proportional amount as found in Prothione™ capsules, and wherein the glycine, L-cystine, and L-glutamine are present as free-form amino acids.

Embodiments of the present invention also relate to methods for post-exposure prophylaxis of HIV infection, especially for patients who are at high risk of being exposed to HIV. Compositions as described herein can be administered as soon as possible after an exposure to a bodily fluid from someone with HIV, or as soon as possible after receiving an HIV diagnosis.

Certain embodiments of the present disclosure relate to prophylactic methods of reducing the ability of a human immunodeficiency virus to infect cells, and of reducing the development of severe disease if a subject becomes infected, comprising administering a composition as described herein. For example, in certain embodiments, a subject exposed to HIV, or at risk of being exposed to HIV, is administered a composition as described herein (e.g., three 1-gram Prothione™ capsules once-daily, or three 1-gram Prothione™ capsules twice daily), for example, as a pre-cautionary measure to reduce the ability of the virus to infect cells and replicate if the subject is exposed to the virus.

### Examples

A randomized, double-blinded, placebo-controlled clinical trial was conducted to evaluate the safety and efficacy of Prothione™ capsules in the treatment of patients with mild to moderate COVID-19. The study included some subjects with HIV, and summarized below are results pertaining to the HIV+ study subjects.

Study subjects were adults≥18 years of age diagnosed with COVID-19 by a standardized RT-PCR assay and having mild to moderate symptoms associated with COVID-19. Patients were deemed to have mild disease if they suffered mild symptoms such as fever, rhinorrhea, mild cough, sore throat, malaise, headache, and/or muscle pain, and did not suffer from shortness of breath or exhibit signs of a more serious lower airway disease (respiratory rate <20 breaths/minute, heart rate <90 beats/minute, and oxygen saturation (pulse oximetry) >93% on room air). Moderate disease included the symptoms above as well as more significant lower respiratory symptoms, such as shortness of breath (at rest or with exertion), and signs of moderate pneumonia but without signs of more serious lower airway disease (respiratory rate ≥20 but <30 breaths/minute, heart rate ≥90 but less than 125 beats/minute, oxygen saturation (pulse oximetry) >93% on room air, and if available, lung infiltrates based on X-ray or CT scan <50% present). Key exclusion criteria were: severe COVID-19 disease; signs of acute respiratory distress syndrome (ARDS) or respiratory failure necessitating mechanical ventilation at the time of screening; and history of systemic corticosteroids. HIV was not an exclusion criterion, and thus subjects with HIV were not excluded from the study.

A total of 231 COVID-19 patients enrolled in the study and were randomized to the treatment arm (115 patients) or placebo arm (116 patients); of these study subjects, a total of ten subjects with HIV were randomized. Approximately half of patients in each arm had four or more COVID-19 symptoms (e.g., fever, dyspnea, cough, and myalgia) at the time of enrollment. During the trial, seven subjects withdrew from the treatment arm and seventeen subjects withdrew from the placebo arm. Due to withdrawals, protocol deviations, and loss to follow-up, 107 patients in the treatment arm and 97 patients in the placebo arm completed the study.

Subjects in the treatment arm were administered three 1-gram Prothione™ capsules (3 grams Prothione™) orally twice a day, for thirty days, for a total daily dose of 6 grams Prothione™. Each Prothione™ capsule contains glycine, L-glutamine, L-cystine, coenzyme Q10, and selenomethionine. Six grams of Prothione™ daily provides a daily dosage of: glycine (2355 mg), L-glutamine (2355 mg), L-cystine (1200.6 mg), coenzyme Q10 (12 mg), and selenomethionine (0.034 mg). Subjects in the placebo arm were administered a placebo twice daily.

Within three days of the initial screening visit, study subjects began the 30-day treatment period. Following the treatment period, subjects had two additional visits for follow-up, at 7 days and 30 days after the last dose.

### Clinical Endpoints Included the Following:

Time (in days) to clinical resolution (TTCR); clinical resolution was reached when a subject had three consecutive negative RT-PCR tests (for SARS-COV-2 virus), each of the last two tests conducted within 24-36 hours of the previous test.
Clinical improvement in fever, dyspnea, cough, and/or myalgia, as determined by, e.g., Clinical Symptom Score Assessment (score 0-12).
Progression of COVID-19 and hospitalizations.
Change from baseline in red blood cell intracellular glutathione levels.
COVID-19 viral load, measured on days 0, 1, 2, 3, 4, 5, 6, 7, 10, 14, 21, and 29 or until a subject had three consecutive negative RT-PCR tests, with each of the last two tests conducted within 24-36 hours of the previous test.
HIV viral load in HIV+positive study subjects.
   Incidence of treatment-related adverse events (TRAEs), and incidence and severity of treatment-emergent adverse events (TEAEs) were monitored, as were changes in blood chemistry, ECG, and vitals. This trial involved 14,091 clinical visits with study subjects.

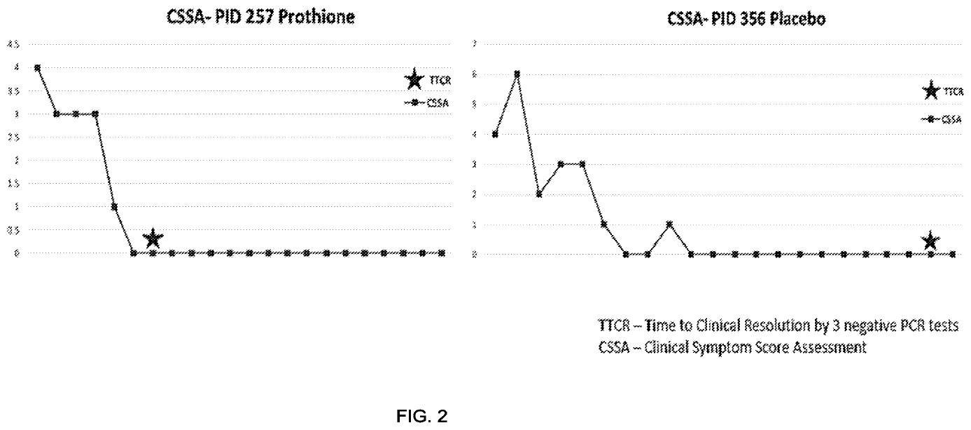

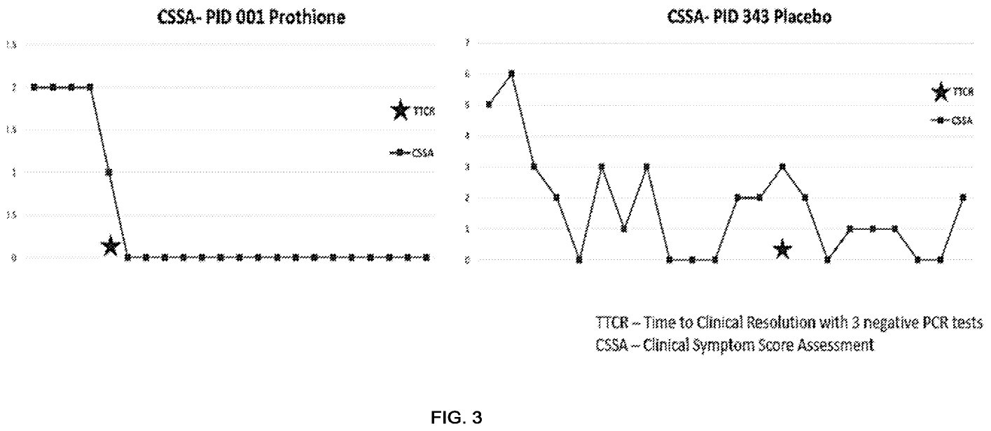

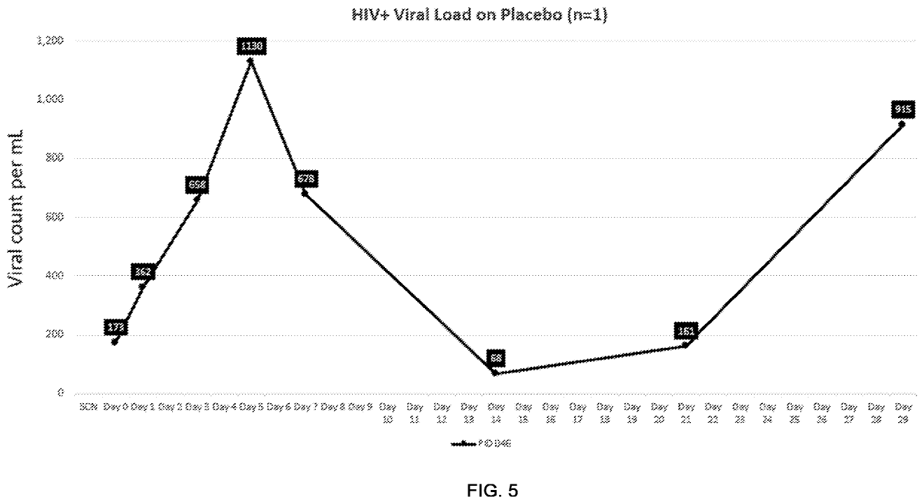

FIGS. 1-5 provide results for HIV+ study subjects. HIV+ study subjects in the Prothione™-treatment arm recovered more quickly compared to HIV+ study subjects in the placebo arm, as shown in FIGS. 1-3. FIGS. 4 and 5 are graphs showing changes in HIV viral load in individual HIV+ study subjects. As shown in FIG. 4, HIV viral load decreased, and for study subject PID 368 dramatically decreased, with the administration of Prothione™ capsules. In contrast, as shown in FIG. 5, such a decrease in HIV viral load was not observed for an HIV+ study subject receiving placebo.

The study also showed that Prothione™ capsules are safe, with no difference in safety measures compared to placebo. There were a total of 25 Treatment Related Adverse Events (TRAEs) during the treatment period. No TRAEs led to discontinuations or withdrawals from the study. In addition, there was no clustering of events for a single system, and many TRAEs were related to incidental diagnoses. No renal signals or abnormal liver enzymes were observed, and no changes in lymphocytes or platelets, lipid metabolism, glucose levels, or electrolytes were observed.

While this invention has been particularly shown and described with references to certain embodiments thereof, it will be understood in light of the present disclosure by those skilled in the art that various changes in form and details may be made therein without departing from the scope of the invention, for example as encompassed by the appended claims.

1. A method for reducing viral load in a subject infected with a human immunodeficiency virus, the method comprising administering to the subject, on a daily basis and for at least three consecutive days, either one of the following:
about 2,355 mg glycine, about 2,355 mg L-glutamine, about 1,200 mg L-cystine, about 12 mg coenzyme Q10, and about 0.034 mg selenomethionine; or
about 1,177 mg glycine, about 1,177 mg L-glutamine, about 600 mg L-cystine, about 6 mg coenzyme Q10, and about 0.017 mg selenomethionine.

2. The method according to claim 1, wherein the about 2,355 mg glycine, about 2,355 mg L-glutamine, about 1,200 mg L-cystine, about 12 mg coenzyme Q10, and about 0.034 mg selenomethionine are administered, and wherein the subject is an adult human.

3. The method according to claim 1, wherein the subject is administered the about 2,355 mg glycine, about 2,355 mg L-glutamine, about 1,200 mg L-cystine, about 12 mg coenzyme Q10, and about 0.034 mg selenomethionine, or the about 1,177 mg glycine, about 1,177 mg L-glutamine, about 600 mg L-cystine, about 6 mg coenzyme Q10, and about 0.017 mg selenomethionine, for at least five consecutive days.

4. (canceled)

5. The method according to claim 1, wherein the about 1,177 mg glycine, about 1,177 mg L-glutamine, about 600 mg L-cystine, about 6 mg coenzyme Q10, and about 0.017 mg selenomethionine are administered, and wherein the subject is a human of at least 3 years of age and weighing no more than about 40 kg

6. (canceled)

7. The method according to claim 1, wherein a composition comprises:
the about 2,355 mg glycine, about 2,355 mg L-glutamine, about 1,200 mg L-cystine, about 12 mg coenzyme Q10, and about 0.034 mg selenomethionine; or the about 1,177 mg glycine, about 1,177 mg L-glutamine, about 600 mg L-cystine, about 6 mg coenzyme Q10, and about 0.017 mg selenomethionine; and
wherein the administering comprises orally administering to the subject the composition once-daily or twice-daily for at least three consecutive days.

8. (canceled)

9. The method according to claim 7, wherein the composition comprises the about 1,177 mg glycine, about 1,177 mg L-glutamine, about 600 mg L-cystine, about 6 mg coenzyme Q10, and about 0.017 mg selenomethionine, and wherein the subject is a human of at least 3 years of age and weighing no more than about 40 kg.

10. A method of preventing a human immunodeficiency virus infection in a subject, the method comprising orally administering to the subject a composition either one of the following:
about 2,355 mg glycine, about 2,355 mg L-glutamine, about 1,200 mg L-cystine, about 12 mg coenzyme Q10, and about 0.034 mg selenomethionine; or
about 1,177 mg glycine, about 1,177 mg 1a-glutamine, about 600 mg L-cystine, about 6 mg coenzyme Q10, and about 0.017 mg selenomethionine.

11. The method according to claim 1, wherein the human immunodeficiency virus is HIV-1.

12. Use of glycine, L-glutamine, L-cystine, coenzyme Q10, and selenomethionine in the preparation of a medicament for treating HIV-1 infection in a subject.

13. (canceled)

14. The method according to claim 7, wherein the composition comprises the about 2,355 mg glycine, about 2,355 mg L-glutamine, about 1,200 mg L-cystine, about 12 mg coenzyme Q10, and about 0.034 mg selenomethionine, and wherein the subject is an adult human.

15. The method according to claim 7, wherein the composition is administered for at least five consecutive days.

16. The method according to claim 10, wherein the composition comprises the about 2,355 mg glycine, about 2,355 mg L-glutamine, about 1,200 mg L-cystine, about 12 mg coenzyme Q10, and about 0.034 mg selenomethionine, and wherein the subject is an adult human.

17. The method according to claim 10, wherein the composition comprises the about 1,177 mg glycine, about 1,177 mg L-glutamine, about 600 mg L-cystine, about 6 mg coenzyme Q10, and about 0.017 mg selenomethionine, and wherein the subject is a human of at least 3 years of age and weighing no more than about 40 kg.

18. The method according to claim 10, wherein the composition is administered to the subject once-daily or twice daily, for at least three consecutive days.

19. The method according to claim 18, wherein the composition is administered for at least five consecutive days.
